(19) United States
(12) Patent Application Publication
(10) Pub. No.: US 2019/0197762 A1
(43) Pub. Date: Jun. 27, 2019

(54) CPR IMAGE GENERATION APPARATUS, METHOD, AND PROGRAM

(71) Applicant: FUJIFILM Corporation, Tokyo (JP)

(72) Inventor: Tsutomu INOUE, Tokyo (JP)

(73) Assignee: FUJIFILM Corporation, Tokyo (JP)

(21) Appl. No.: 16/286,424

(22) Filed: Feb. 26, 2019

Related U.S. Application Data
(63) Continuation of application No. PCT/JP2017/024637, filed on Jul. 5, 2017.

(30) Foreign Application Priority Data
Aug. 31, 2016 (JP) 2016-169176

(51) Int. Cl. G06T 15/08; G06T 19/00; A61B 6/03

(52) U.S. Cl. CPC G06T 15/08; G06T 19/00; A61B 6/037; G06T 2207/30101; G06T 2210/41; G06T 2219/008; G06T 2215/06

(57) ABSTRACT

In a CPR image generation apparatus, method, and program, a CPR image is generated so as to include a region of interest, such as a branch, a myocardium, a plaque, and a lesion. A structure extraction unit 22 extracts a coronary artery region 30 from a three-dimensional image G0. A cross section setting unit 23 sets a cross section Pk perpendicular to a reference line L0 for each point on the reference line L0 in the coronary artery region 30. In a case where a region of interest is present on each cross section Pk, a cutting plane determination unit 24 determines one cutting plane Ck in each cross section including the reference line L0 and the region of interest. An image generation unit 25 generates a CPR image G10 including the cutting plane Ck.

### CROSS REFERENCE TO RELATED APPLICATIONS

This application is a Continuation of PCT International Application No. PCT/JP2017/024637 filed on Jul. 5, 2017, which claims priority under 35 U.S.C § 119(a) to Patent Application No. 2016-169176 filed in Japan on Aug. 31, 2016, all of which are hereby expressly incorporated by reference into the present application.

### BACKGROUND OF THE INVENTION

### 1. Field of the Invention

The present invention relates to a CPR image generation apparatus, method, and program for generating a curved planar reconstruction (CPR) image showing a cross section along the longitudinal direction of a structure, such as a blood vessel.

### 2. Description of the Related Art

Conventionally, in order to observe a lesion of a tubular structure having a lumen, such as a blood vessel, an intestine, a bronchus, and an artery of a subject, a medical image display device is known that has a mode in which an image of a three-dimensional structure can be acquired by volume rendering from a three-dimensional image of an object obtained by a computed tomography (CT) apparatus, a magnetic resonance imaging (MRI) apparatus, or the like and the inner surface of the lumen can be observed as if the operator enters the lumen by simulating the endoscope from the three-dimensional image. Maximum intensity projection (MIP) processing, minimum intensity projection (MinIP) processing, multi planar reconstruction (MPR) processing, curved planar reconstruction (CPR) processing, and the like are known as three-dimensional image processing. In particular, the CPR processing is to generate a CPR image by designating a certain curved plane in a three-dimensional image and reconstructing the three-dimensional image along the designated curved plane into a two-dimensional image. In this manner, for a form of the inner wall surface of the tubular structure, it is possible to display a cross section in the longitudinal direction on one screen (for example, refer to JP2007-135843A).

In addition to the three-dimensional image obtained from the above-described CT apparatus or the like, a functional image showing the function of the subject is also used. As the functional image, for example, a SPECT image acquired by single photon emission tomography and a PET image acquired by positron emission tomography are known. By displaying such a functional image together with the CPR image, for example, in a case where a coronary artery is a diagnostic target, it is possible to simultaneously check the state of the coronary artery and the state of the function of the heart.

On the other hand, in a tubular structure extending while meandering, such as a blood vessel, the longitudinal cross section of the tubular structure shown in the CPR image is a cutting curved plane having a curved shape or a twisted shape along the meandering of the tubular structure. For this reason, it has been difficult for a user unfamiliar with the CPR image to determine which curved plane, which is obtained by cutting each position of the tubular structure in a certain direction, the CPR image shows. For this reason, a method of indicating a positional relationship between a cutting curved plane and a tubular structure by displaying an index indicating a longitudinal cross section together with a CPR image has been proposed (refer to JP2012-024517A). In addition, JP2012-024517A has proposed a method of generating a multipath CPR image including all branches in a tubular structure including branches, such as a blood vessel and a bronchus. The method of generating a multipath CPR image described in JP2012-024517A is a method of setting a cutting curved plane for each section between branches and generating a CPR image of each section on one image. The cutting curved plane is set based on information, such as the normal direction of each position of the cutting curved plane. Therefore, since a CPR image including the structure of each branch of the tubular structure can be generated, it is possible to display the structure of the branch of the tubular structure on one screen.

### SUMMARY OF THE INVENTION

In the method described in JP2012-024517A, however, the cutting curved plane is not continuous at the branch position of the tubular structure. Therefore, in the generated multipath CPR image, a boundary appears in the image at the branch position.

On the other hand, diseases, such as myocardial infarction, occur since a stenosed portion of the coronary artery is clogged with thrombus and the blood does not flow downstream of the clogged coronary artery and as a result, the myocardium stops working. The possibility of occurrence of myocardial infarction or the like increases as the stenosis ratio of the blood vessel increases. The cause of the narrowing of the blood vessel is the plaque formed on the blood vessel wall. Therefore, it is very important to observe how much plaque is generated in blood vessels in a CPR image. In order to check lesions including a tumor in the lung, such as a lung cancer, biopsy of a lesion may be performed by inserting an endoscope into the bronchus. In this case, it is important to observe a path to reach the lesion and the relationship between the bronchus and the position of the lesion in the CPR image.

However, regions of interest, such as the myocardium, the plaque, and the lesion, are not present on only one longitudinal cross section of the tubular structure but present at all radial positions centered on a core line in a cross section perpendicular to the center line (hereinafter, referred to as a core line) of the tubular structure connecting the centers or the centers of gravity of respective cross sections of the tubular structure. For this reason, in the case of generating a CPR image by setting only one longitudinal cutting plane as in the related art, a region of interest may not be able to be satisfactorily observed in the CPR image.

The present invention has been made in view of the above circumstances, and it is an object of the present invention to generate a CPR image so as to include a region of interest, such as a branch, a myocardium, a plaque, and a lesion.

A CPR image generation apparatus according to the present invention comprises: structure extraction means for extracting a target structure from a three-dimensional image acquired by imaging; cross section setting means for setting a cross section perpendicular to a reference line of the target structure at each point on the reference line; cutting plane determination means for determining one cutting plane in each cross section, which includes the reference line and a region of interest, in a case where the region of interest is present on each cross section; and image generation means for generating a CPR image including the cutting plane in each cross section.

The CPR image is an image showing a cross section along the core line of the target structure. However, in a case where the target structure includes a plurality of branches as in, for example, a blood vessel or a bronchus, a plurality of core lines are present for each branch. In such a case, in order to generate a CPR image, it is necessary to determine one core line. The “reference line” means one core line for generating a CPR image in a case where the target structure includes a plurality of core lines. In a case where the target structure includes only one core line, the one core line is the reference line.

In the CPR image generation apparatus according to the present invention, the target structure may be a tubular structure.

In the CPR image generation apparatus according to the present invention, the region of interest may be at least one of a branch of the target structure, other structures adjacent to the target structure, other structures within the target structure, or a lesion.

As “other structures adjacent to the target structure”, for example, a myocardium in a case where a coronary artery is the target structure can be mentioned. As “other structures within the target structure”, for example, a plaque formed in a stenosed portion in a case where a coronary artery is the target structure can be mentioned.

In the CPR image generation apparatus according to the present invention, the image generation means may generate a cutting curved plane on which the cutting plane in each cross section is continuous and generate the CPR image including the cutting curved plane.

In the CPR image generation apparatus according to the present invention, in a case where a plurality of regions of interest are present in each cross section, the cutting plane determination means may select one region of interest.

The CPR image generation apparatus according to the present invention may further comprise display means for displaying the CPR image.

In this case, the display means may display information indicating an angle of the cutting plane in each cross section centered on the reference line from a cutting plane of a reference cross section.

A CPR image generation method according to the present invention comprises: extracting a target structure from a three-dimensional image acquired by imaging; setting a cross section perpendicular to a reference line of the target structure at each point on the reference line; determining one cutting plane in each cross section, which includes the reference line and a region of interest, in a case where the region of interest is present on each cross section; and generating a CPR image including the cutting plane in each cross section.

In addition, a program causing a computer to execute the CPR image generation method according to the present invention may be provided.

According to the present invention, a target structure and the reference line of the target structure are extracted from the three-dimensional image, and a cross section perpendicular to a reference line is set at each point on the reference line. Then, in a case where a region of interest is present in each cross section, one cutting plane in each cross section including a region of interest relevant to the reference line and the target structure is determined, and a CPR image including the one cutting plane in each cross section is generated. For this reason, it is possible to generate a CPR image so as to include regions of interest present in all radial directions around the reference line in each cross section. Therefore, it is possible to satisfactorily observe a plurality of regions of interest included in the target structure in the CPR image. In addition, since one cutting plane is determined in each cross section, for example, in a case where the region of interest is a branch, it is possible to include a plurality of branches in the CPR image, and the CPR image is continuous between branches. Therefore, it is possible to generate a CPR image of a natural impression with no boundary.

### BRIEF DESCRIPTION OF THE DRAWINGS

FIG. 1 is a hardware configuration diagram showing an outline of a diagnostic support system to which a CPR image generation apparatus according to an embodiment of the present invention is applied.

FIG. 2 is a schematic block diagram showing the configuration of the CPR image generation apparatus according to the present embodiment.

FIG. 3 is a diagram showing an extracted coronary artery region.

FIG. 4 is a diagram illustrating a region where a plaque is present.

FIG. 5 is a diagram illustrating a region where a myocardium is present.

FIG. 6 is a diagram illustrating a region where a branch is present.

FIG. 7 is a diagram illustrating the generation of a cutting curved plane.

FIG. 8 is a diagram illustrating the generation of a cutting curved plane.

FIG. 9 is a diagram showing a CPR image.

FIG. 10 is a flowchart showing a process performed in the present embodiment.

FIG. 11 is a diagram illustrating an angle centered on a reference line from the cutting plane of a cross section as a reference.

FIG. 12 is a diagram showing a CPR image displaying angle information.

### DESCRIPTION OF THE PREFERRED EMBODIMENTS

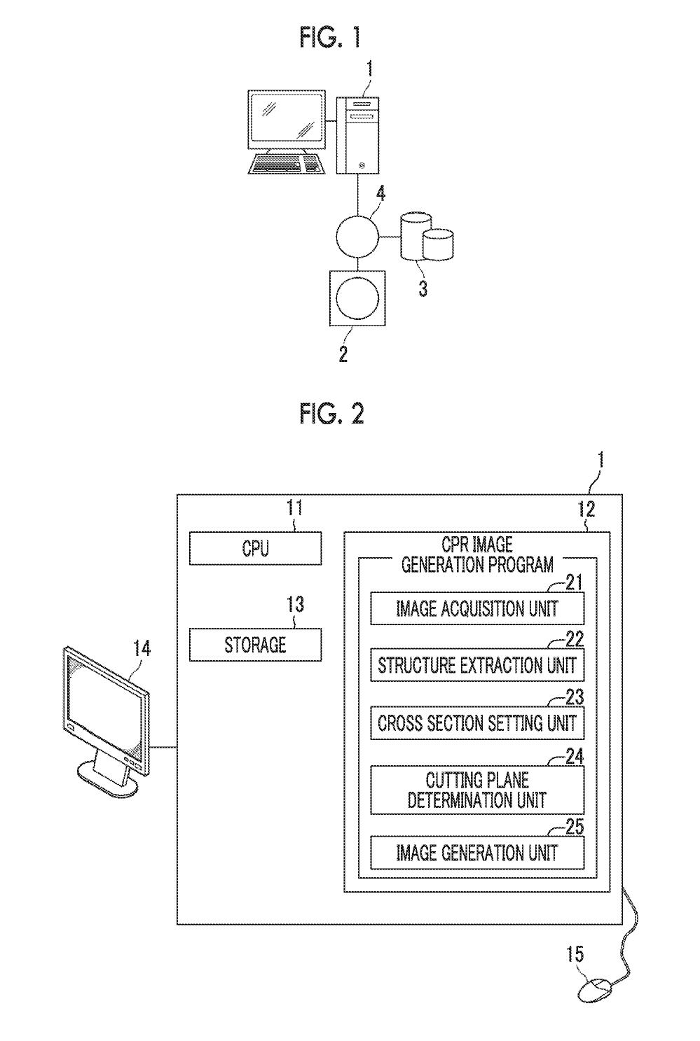

Hereinafter, an embodiment of the present invention will be described with reference to the accompanying diagrams. FIG. 1 is a hardware configuration diagram showing the outline of a diagnostic support system to which a CPR image generation apparatus according to a first embodiment of the present invention is applied. As shown in FIG. 1, in the diagnostic support system, a CPR image generation apparatus 1 according to the present embodiment, a three-dimensional image capturing apparatus 2, and an image storage server 3 are communicably connected to each other through a network 4. Then, in the diagnostic support system, a CPR image of a part as a diagnostic target of a subject is generated in the CPR image generation apparatus 1.

The three-dimensional image capturing apparatus 2 is an apparatus that generates a three-dimensional image showing a diagnostic target part of a subject by imaging the part. Specifically, the three-dimensional image capturing apparatus 2 is a CT apparatus, an MRI apparatus, a positron emission tomography (PET) apparatus, or the like. The three-dimensional image generated by the three-dimensional image capturing apparatus 2 is transmitted to the image storage server 3 and stored therein. In the present embodiment, it is assumed that the diagnostic target part of the subject is a coronary artery, the three-dimensional image capturing apparatus 2 is a CT apparatus, and a three-dimensional image of the chest of the subject is generated.

The image storage server 3 is a computer that stores and manages various kinds of data, and comprises a large-capacity external storage device and software for database management. The image storage server 3 communicates with other devices through the wired or wireless network 4 to transmit and receive image data or the like. Specifically, the image storage server 3 acquires image data, such as a three-dimensional image generated by the three-dimensional image capturing apparatus 2, through the network, and stores the image data in a recording medium, such as a large-capacity external storage device, to manage the image data. The storage format of image data and the communication between devices through the network 4 are based on a protocol, such as a digital imaging and communication in medicine (DICOM).

The CPR image generation apparatus 1 is realized by installing a CPR image generation program of the present invention on one computer. The computer may be a workstation or a personal computer that is directly operated by a doctor who performs diagnosis, or may be a server computer connected to these through a network. The CPR image generation program is distributed in a state in which the CPR image generation program is recorded on a recording medium, such as a digital versatile disc (MID) or a compact disk read only memory (CD-ROM), and is installed onto the computer from the recording medium. Alternatively, the CPR image generation program is stored in a storage device of a server computer connected to the network or in a network storage so as to be accessible from the outside, and is downloaded and installed onto a computer used by a doctor as necessary.

FIG. 2 is a diagram showing the schematic configuration of a CPR image generation apparatus realized by installing a CPR image generation program on a computer. As shown in FIG. 2, the CPR image generation apparatus I comprises a central processing unit (CPU) 11, a memory 12, and a storage 13 as the configuration of a standard workstation. A display 14 and an input unit 15, such as a mouse, are connected to the CPR image generation apparatus 1.

Latest three-dimensional images and past three-dimensional images at the time of previous diagnosis for the same subject, which are acquired from the image storage server 3 through the network 4, and various kinds of information including information required for processing are stored in the storage 13. In the present embodiment, it is assumed that a three-dimensional image G0 having the chest of the same subject as a target part is stored.

A CPR image generation program is stored in the memory 12. As processing to be executed by the CPU 11, the CPR image generation program defines: image acquisition processing for acquiring the three-dimensional image G0 acquired by the three-dimensional image capturing apparatus 2; structure extraction processing for extracting a target structure from the three-dimensional image G0; cross section setting processing for setting a cross section perpendicular to a reference line, which is one of the core lines of the target structure, at each point on the reference line; cutting plane determination processing for determining one cutting plane in each cross section including the reference line and a region of interest in a case where a region of interest is present on each cross section; and image generation processing for generating a CPR image including the cutting plane in each cross section.

Then, the CPU 11 executes these processes according to the program, so that the computer functions as an image acquisition unit 21, a structure extraction unit 22, a cross section setting unit 23, a cutting plane determination unit 24, and an image generation unit 25. The CPR image generation apparatus 1 may include a plurality of processors or processing circuits that perform image acquisition processing, structure extraction processing, cross section setting processing, cutting plane determination processing, and image generation processing.

The image acquisition unit 21 acquires the three-dimensional image G0 of the chest including a coronary artery, that is, a heart, which is a diagnostic target part, from the image storage server 3. In a case where the three-dimensional image G0 is already stored in the storage 13, the image acquisition unit 21 may acquire the three-dimensional image G0 from the storage 13.

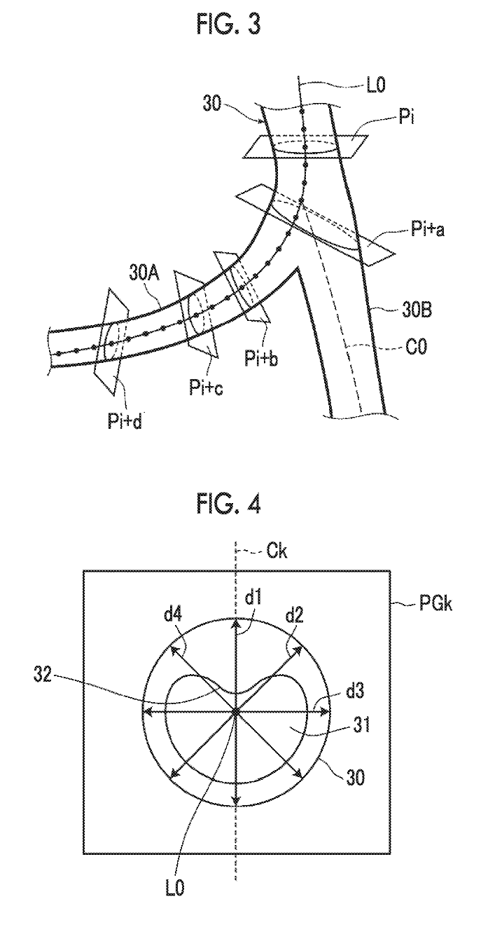

The structure extraction unit 22 extracts a coronary artery region from the three-dimensional image G0 using the methods described in JP2010-200925A and JP2010-220742A, for example. In this method, first, based on the value of voxel data forming volume data, the positions and main axis directions of a plurality of candidate points forming the core line of the coronary artery are calculated. Alternatively, by calculating a Hessian matrix for the volume data and analyzing the eigenvalues of the calculated Hessian matrix, the position information and the main axis directions of a plurality of candidate points forming the core line of the coronary artery are calculated. Then, a feature amount indicating the likelihood of coronary artery is calculated for voxel data around the candidate points, and it is determined whether or not the voxel data indicates a coronary artery region based on the calculated feature amount. Determination based on the feature amount is performed based on an evaluation function acquired in advance by machine learning. As a result, a coronary artery region 30 is extracted from the volume data. FIG. 3 shows a part of the extracted coronary artery region 30.

The cross section setting unit 23 sets a cross section perpendicular to the reference line at each point on the reference line of the coronary artery region 30. In the methods described in JP2010-200925A and JP2010-2207424, the core line of the coronary artery is set in the process of extracting the coronary artery region 30. For each candidate point forming the core line, the position and the main axis direction are calculated. Therefore, at each candidate point, a cross section (orthogonal cross section) perpendicular to the main axis direction can be set as a cross section perpendicular to the core line.

Here, since the coronary artery region 30 extracted in the present embodiment has a branch as shown in FIG. 3, the core line is divided into two parts at the branch. In order to generate a CPR image, it is necessary to determine one core line. For this reason, the cross section setting unit 23 determines a core line for generating a CPR image between the two core lines as a reference line, and sets a cross section perpendicular to the reference line in the reference line. The reference line is determined by displaying the extracted coronary artery region on the display 14 and receiving an instruction from the input unit 15 of the operator. In the present embodiment, it is assumed that the core line of a coronary artery region 30A extending from the upper side to the left side in FIG. 3 is determined as a reference line L0. In FIG. 3, the reference line L0 is shown by a solid line. In FIG. 3, a core line C0 of a coronary artery region 30B extending downward from the branch is shown by a broken line. In FIG. 3, candidate points on the reference line L0 are shown by black dots.

The cross section setting unit 23 sets a cross section perpendicular to the reference line L0 at each point, that is, each candidate point, on the reference line L0. FIG. 3 shows a state in which five cross sections Pi, Pi+a, Pi+b, Pi+c, Pi+d are set by thinning out the candidate points for description.

In a case where there is a region of interest on each cross section Pk (k=1 to n: n is the number of candidate points) set for the reference line L0 by the cross section setting unit 23, the cutting plane determination unit 24 determines one cutting plane Ck in each cross section Pk including the reference line L0 and the region of interest. Therefore, the cutting plane determination unit 24 sets a region of interest in each cross section Pk. The region of interest can be a region of a myocardium 33 and a region where a plaque 32 is formed in each cross section Pk. At the position of a branch 34 in the coronary artery region 30, the branch 34 can be a region of interest. In order to set a region of interest, the cutting plane determination unit 24 generates a cross-sectional image PGk of each cross section Pk from the three-dimensional image G0.

Here, as shown in FIG. 4, the cutting plane determination unit 24 calculates diameters d1 to d4 of lumens 31 of a plurality of coronary artery regions 3C) (here, in four directions) around the reference line L0 in the cross-sectional image PG-k. Here, in a case where the plaque 32 is formed in the lumen 31 of the coronary artery, the diameter of the lumen 31 becomes narrow. For this reason, the cutting plane determination unit 24 sets the position of the lumen 31 having a minimum diameter, among the diameters d1 to d4, as a region of interest where the plaque 32 is present. In FIG. 4, the position of the lumen 31 having the diameter d1 is set as a region of interest.

Since the coronary artery is present on the surface of the heart, the cross-sectional image PGk includes the myocardium 33 as shown in FIG. 5. The activity status of the myocardium 33 changes depending on whether or not a sufficient blood flow is supplied to the coronary artery. Therefore, the myocardium 33 is an important region for diagnosis of myocardial infarction or the like. The cutting plane determination unit 24 determines, as a region of interest, a position where the myocardium 33 is present in the cross-sectional image of each cross section Pk.

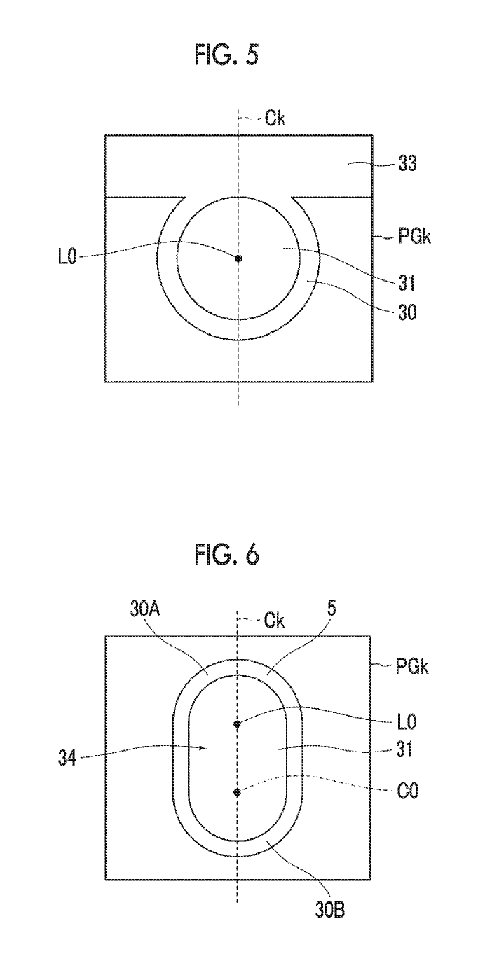

In a case where the cross section Pk is present in the branch, the cutting plane determination unit 24 sets the branch 34 as a region of interest, as shown in FIG. 6.

Then, the cutting plane determination unit 24 determines a cutting plane in each cross section Pk so as to include the region of interest. For example, in a case where the plaque 32 is a region of interest as shown in FIG. 4, the cutting plane Ck is determined so as to include the diameter d1. In a case where the myocardium 33 is a region of interest as shown in FIG. 5, the cutting plane Ck is determined so as to pass through the reference line L0 and include the myocardium 33. In a case where the branch 34 is a region of interest as shown in FIG. 6, the cutting plane Ck is determined so as to pass through the core line C0 that is not the reference line L0.

Depending on the position of the cross section Pk, there may be a plurality of regions of interest, such as a plaque and a myocardium or a plaque and a branch. In particular, in the present embodiment, since the coronary artery is a diagnostic target structure, a myocardium is necessarily included in each cross section Pk. In the present embodiment, the cutting plane determination unit 24 sets a priority in advance for each region of interest, selects a region of interest having a high priority in a case where a plurality of regions of interest are present in one cross section, and determines a cutting plane so as to include the selected region of interest. In the present embodiment, it is assumed that priorities are set in the order of plaque, branch, and myocardium. Therefore, in a region where the plaque 32 is present in the coronary artery region 30, the plaque 32 is selected as a region of interest even in a case where the myocardium 33 is present. In addition, in a region where the branch 34 is present in the coronary artery region 30, the branch 34 is selected as a region of interest even in a case where the myocardium 33 is present. Alternatively, the cross-sectional image PGk of each cross section Pk may be displayed on the display 14, and the operator may select a region of interest included in the cutting plane.

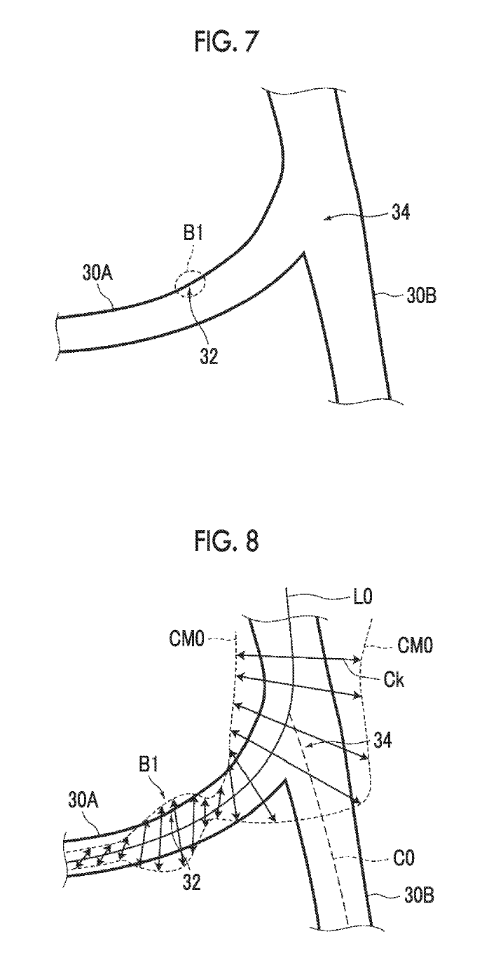

The image generation unit 25 generates a cutting curved plane by smoothly connecting the cutting plane Ck of each cross section Pk determined by the cutting plane determination unit 24 by interpolation or the like. FIGS. 7 to 8 are diagrams illustrating the generation of a cutting curved plane. In the present embodiment, it is assumed that the myocardium 33 is present behind the coronary artery region 30 and as shown in FIG. 7, the plaque 32 is present in a portion B1 surrounded by a broken line of the coronary artery region 30A. In this case, as shown in FIG. 8, a cutting curved plane CM0 is determined by the cutting plane determination unit 24 so that a plurality of regions of interest in each cross section Pk are cut. In FIG. 8, the direction of the arrow is the direction of the cutting plane Ck in each cross section Pk. As a result, the cutting curved plane CM0 is determined so as to cut the plaque 32 in the portion B1, cut the coronary artery region 30B in the branch 34, and cut the myocardium 33 in the other region.

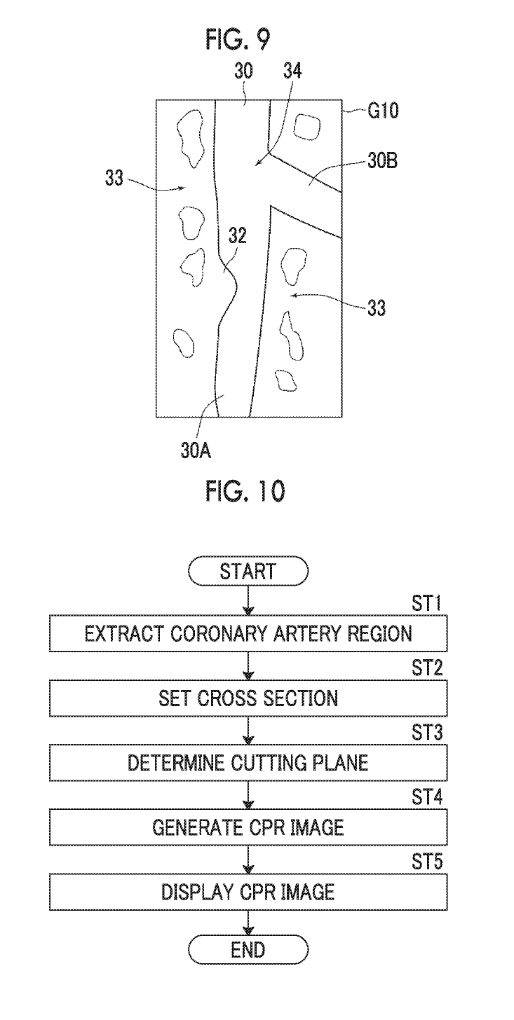

Then, the image generation unit 25 generates a CPR image by cutting the coronary artery region 30 with the cutting curved plane CM0. The CPR image generated in the present embodiment is a straightened CPR image, but a stretched CPR image or a projected CPR image may be used. FIG. 9 is a diagram showing a CPR image. As shown in FIG. 9, in a CPR image G10 generated in the present embodiment, the plaque 32 and the branch 34 are included in the coronary artery region 30. Since the branch 34 is included, the coronary artery region 30B for the core line C0, which is not the reference line L0, is included. In the CPR image G10, a portion other than the plaque 32 and the coronary artery region 30B is the myocardium 33.

Next, a process performed in the present embodiment will be described. FIG. 10 is a flowchart showing the process performed in the present embodiment. First, the image acquisition unit 21 acquires the three-dimensional image G0, and the structure extraction unit 22 extracts the coronary artery region 30 from the three-dimensional image G0 (step ST1). Then, the cross section setting unit 23 sets the cross section Pk perpendicular to the reference line L0 for each point on the reference line L0 in the coronary artery region 30 (step ST2). Then, in a case where a region of interest is present on each cross section Pk, the cutting plane determination unit 24 determines one cutting plane Ck in each cross section including the reference line L0 and the region of interest (step ST3). Then, the image generation unit 25 generates the CPR image G10 including the cutting plane Ck (step ST4), displays the generated CPR image G10 on the display 14 (step ST5), and end the process.

As described above, according to the present embodiment, it is possible to generate the CPR image G10 so as to include regions of interest present in all radial directions around the reference line in each cross section. Therefore, it is possible to satisfactorily observe a plurality of regions of interest included in the coronary artery region 30 in the CPR image G10. In addition, since one cutting plane Ck is determined in each cross section Pk, for example, in a case where the region of interest is a branch, it is possible to include a plurality of branches in the CPR image, and the CPR image G10 is continuous at the branch position. Therefore, it is possible to generate the CPR image G10 of a natural impression with no boundary.

By generating the cutting curved plane CM0 on which the cutting plane Ck in each cross section Pk is continuous and generating the CPR image G10 including the cutting curved plane CM0, the cutting plane Ck changes smoothly. Therefore, it is possible to prevent the occurrence of a boundary between the cross sections in the CPR image G10.

In a case where a plurality of regions of interest are present in each cross section, one region of interest is selected, so that the selected region of interest can be included in the CPR image G10.

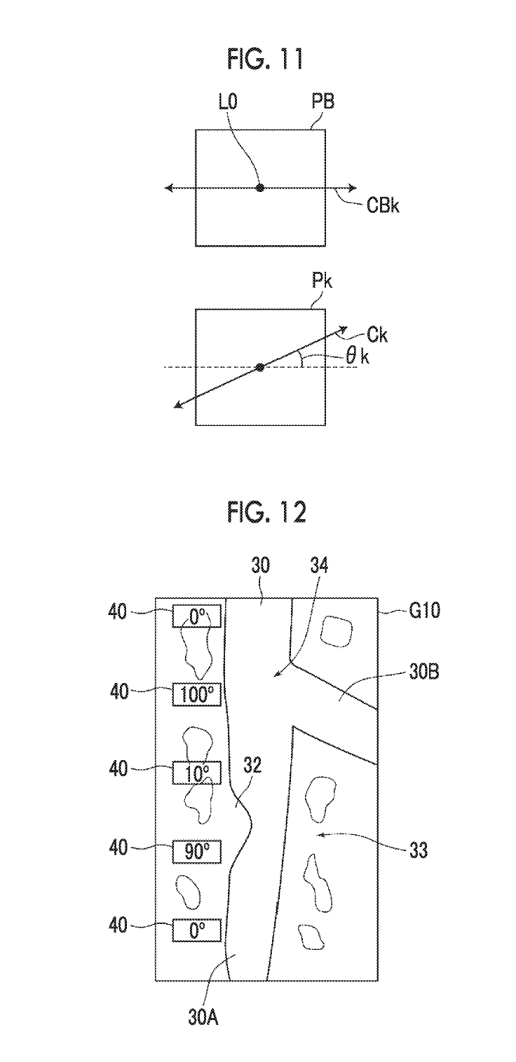

In the embodiment described above, in the case of displaying the CPR image G10, information indicating the angle of the cutting plane Ck in each cross section Pk centered on the reference line L0 from the cutting plane of the reference cross section may be displayed. For example, a cutting plane CBk in a cross section PB as a reference shown on the upper side of FIG. 11 is set as a reference cutting plane. Then, for the cutting plane Ck of a certain cross section Pk shown on the lower side of FIG. 11 an angle θk centered on the reference line L0 from the cutting plane CBk as a reference is calculated. Then, angle information indicating the calculated angle θk is displayed so as to be associated with the cross section Pk from which the cutting plane Ck is obtained in the CPR image G10. FIG. 12 is a diagram showing a CPR image displaying angle information 40. The angle information 40 is displayed so as to he associated with the appropriately sampled cross section Pk. Therefore, it is possible to recognize how much angle the region of interest in the CPR image G10 deviates from the reference cutting plane. The coronary artery region 30 in the CPR image G10 may be displayed in a color-coded manner according to the magnitude of the angle θk.

In the embodiment described above, the coronary artery is a target structure. However, other tubular structures, for example, a bronchus and a large intestine may be used as target structures. In a case where the target structure is a bronchus, the structure extraction unit 22 extracts the structure of the bronchus from the three-dimensional image G0 as a bronchial region. Specifically, a graph structure of a bronchial region included in the input three-dimensional image G0 is extracted as a three-dimensional bronchial region using the method described in JP2010-220742A or the like, for example. In a case where the target structure is a large intestine, the structure extraction unit 22 extracts, as a large intestine region, a region where the pixel value of the large intestine is obtained in the three-dimensional image G0.

In a case where the bronchus is a target structure, a tumor present in the vicinity of the bronchus is important for diagnosis. In a case where the large intestine is a target structure, a tumor present in the luminal wall of the large intestine is important for diagnosis. Therefore, in the structure extraction unit 22, it is preferable to extract a tumor by computer-aided diagnosis (CAD). In this case, the cross section setting unit 23 may set the cutting plane Ck with the tumor as a region of interest.

In particular, according to the present embodiment, in a case where the bronchus is a target structure, the CPR image G10 can be generated so as to include a plurality of branches and tumors from the entrance of the bronchus. Therefore, in the case of performing an endoscopic examination for biopsy of a tumor, it is possible to easily check through which branch of the bronchus the endoscope can reach the tumor by using the CPR image G10.

In the embodiment described above, a CPR image is generated for a tubular structure, such as a coronary artery. However, the present invention is not limited thereto, and a CPR image may be generated using a structure extending in the longitudinal direction, such as the spinal column and limbs, as a target structure.

In the embodiment described above, a functional image of the myocardium may be displayed simultaneously with the CPR image G10. As a result, it is possible to simultaneously check the state of the coronary artery and the state of the function of the heart.

Hereinafter, the effect of the present embodiment will be described.

Since the cutting plane is smoothly changed by generating the cutting curved plane on which the cutting plane in each cross section is continuous and generating the CPR image including the cutting curved plane, it is possible to more reliably prevent the occurrence of a boundary between the cross sections in the CPR image.

In a case where a plurality of regions of interest are present in each cross section, one region of interest is selected, so that the selected region of interest can be included in the CPR image.

By displaying the information indicating the angle of the cutting plane in each cross section centered on the reference line from the cutting plane of the reference cross section, it is possible to recognize how much angle the region of interest in the CPR image deviates from the cutting plane of the cross section as a reference.

### EXPLANATION OF REFERENCES

1: CPR image generation apparatus

2: three-dimensional image capturing apparatus

3: image storage server

4: network

11: CPU

12: memory

13: storage

14: display

15: input unit

21: image acquisition unit

22: structure extraction unit

23: cross section setting unit

24: cutting plane determination unit

25: image generation unit

30, 30A, 30B: coronary artery region

31: lumen

32: plaque

33: myocardium

34: branch

40: angle information

Ck: cutting plane

CM0: cutting curved plane

G10: CPR image

Pk: cross section

PGk: cross-sectional image

What is claimed is:

1. A CPR image generation apparatus, comprising:
a structure extraction unit that extracts a target structure from a three-dimensional image acquired by imaging;
a cross section setting unit that sets a cross section perpendicular to a reference line of the target structure at each point on the reference line;
a cutting plane determination unit that determines one cutting plane in each cross section, which includes the reference line and a region of interest, in a case where the region of interest is present on each cross section; and
an image generation unit that generates a CPR image including the cutting plane in each cross section.

2. The CPR image generation apparatus according to claim 1,
wherein the target structure is a tubular structure.

3. The CPR image generation apparatus according to claim 1,
wherein the region of interest is at least one of a branch of the target structure, other structures adjacent to the target structure, other structures within the target structure, or a lesion.

4. The CPR image generation apparatus according to claim 2,
wherein the region of interest is at least one of a branch of the target structure, other structures adjacent to the target structure, other structures within the target structure, or a lesion.

5. The CPR image generation apparatus according to claim 1,
wherein the image generation unit generates a cutting curved plane on which the cutting plane in each cross section is continuous, and generates the CPR image including the cutting curved plane.

6. The CPR image generation apparatus according to claim 2,
wherein the image generation unit generates a cutting curved plane on which the cutting plane in each cross section is continuous, and generates the CPR image including the cutting curved plane.

7. The CPR image generation apparatus according to claim 3,
wherein the image generation unit generates a cutting curved plane on which the cutting plane in each cross section is continuous, and generates the CPR image including the cutting curved plane.

8. The CPR image generation apparatus according to claim 1,
wherein, in a case where a plurality of regions of interest are present in each cross section, the cutting plane determination unit selects one region of interest.

9. The CPR image generation apparatus according to claim 2,
wherein, in a case where a plurality of regions of interest are present in each cross section, the cutting plane determination unit selects one region of interest.

10. The CPR image generation apparatus according to claim 3,
wherein, in a case where a plurality of regions of interest are present in each cross section, the cutting plane determination unit selects one region of interest.

11. The CPR image generation apparatus according to claim 4,
wherein, in a case where a plurality of regions of interest are present in each cross section, the cutting plane determination unit selects one region of interest.

12. The CPR image generation apparatus according to claim 1, further comprising:
display that displays the CPR image.

13. The CPR image generation apparatus according to claim 2, further comprising:
display that displays the CPR image.

14. The CPR image generation apparatus according to claim 3, further comprising:
display that displays the CPR image.

15. The CPR image generation apparatus according to claim 4, further comprising:
display that displays the CPR image.

16. The CPR image generation apparatus according to claim 12,
wherein the display displays information indicating an angle of the cutting plane in each cross section centered on the reference line from a cutting plane of a reference cross section.

17. The CPR image generation apparatus according to claim 13,
wherein the display displays information indicating an angle of the cutting plane in each cross section centered on the reference line from a cutting plane of a reference cross section.

18. The CPR image generation apparatus according to claim 14,
wherein the display displays information indicating an angle of the cutting plane in each cross section centered on the reference line from a cutting plane of a reference cross section.

19. A CPR image generation method, comprising:
extracting a target structure from a three-dimensional image acquired by imaging;
setting a cross section perpendicular to a reference line of the target structure at each point on the reference line;
determining one cutting plane in each cross section, which includes the reference line and a region of interest, in a case where the region of interest is present on each cross section; and
generating a CPR image including the cutting plane in each cross section.

20. A non-transitory computer readable recording medium storing a CPR image generation program causing a computer to execute:
a step of extracting a target structure from a three-dimensional image acquired by imaging;
a step of setting a cross section perpendicular to a reference line of the target structure at each point on the reference line;
a step of determining one cutting plane in each cross section, which includes the reference line and a region of interest, in a case where the region of interest is present on each cross section; and
a step of generating a CPR image including the cutting plane in each cross section.
